(19) United States
(12) Patent Application Publication
(10) Pub. No.: US 2018/0168988 A1
(43) Pub. Date: Jun. 21, 2018

(54) AGENT AND METHOD FOR TEMPORARY DEFORMATION OF KERATINOUS FIBERS

(71) Applicant: Henkel AG & Co. KGaA, Duesseldorf (DE)

(72) Inventors: Julia Bibiane Lange, Bad Bramstedt (DE); Rene Scheffler, Ellerau (DE); Diane Metten, Hamburg (DE); Cyrielle Martinez, Hamburg (DE)

(73) Assignee: Henkel AG & Co. KGaA, Duesseldorf (DE)

(21) Appl. No.: 15/790,063

(22) Filed: Oct. 23, 2017

(30) Foreign Application Priority Data
Dec. 19, 2016 (DE) 10 2016 225 467.7

(51) Int. Cl. A61K 8/81; A61Q 5/06

(52) U.S. Cl. CPC A61K 8/8152; A61Q 5/06; A61K 8/8176; A61K 8/8147

(57) ABSTRACT

The present disclosure relates to a cosmetic preparation for temporary deformation of hair, which contains a combination of two specific crosslinked copolymers. The cosmetic preparation provides a good hold.

### CROSS-REFERENCE TO RELATED APPLICATION

This application claims priority to German Patent Application No. 10 2016 225 467.7, filed Dec. 19, 2016, which is incorporated herein by reference in its entirety.

### TECHNICAL FIELD

The present disclosure relates to a cosmetic composition for hair fixing or for temporarily deforming keratinous fibers, such as human hair, wherein the composition contains a combination of two specific crosslinked copolymers.

### BACKGROUND

The temporary design of hairstyles for a longer period of time of up to several days normally requires the use of firming active ingredients. Therefore, hair treatment agents that temporarily shape the hair play an important role. Corresponding agents for temporary deformation normally contain synthetic polymers and/or waxes as a firming active ingredient. Agents aiding the temporary deformation of keratinous fibers can normally be packaged, for instance, as hairspray, hair wax, hair gel or mousse.

A feature of an agent for temporary deformation of hair, also referred to hereinafter as a styling agent, includes giving the treated fibers in the newly modeled shape—i.e. a shape impressed on the hair—a strong hold. This is also referred to as a strong hairstyle hold or a high degree of hold of the styling agent. The hairstyle hold is determined at least in part by the type and amount of firming active ingredients used, wherein the other components of the styling agent can also have an influence.

In addition to a high degree of hold, the styling agent may fulfill a series of additional requirements. They can be roughly divided into characteristics on the hair, characteristics of the respective formulation, e.g. characteristics of the foam, gel or sprayed aerosol, and characteristics relating to the handling of the styling agent, with particular focus in some instances given to the characteristics on the hair. This includes, in particular, moisture resistance, low stickiness (tack) and a balanced conditioning effect. Furthermore, a styling agent should be universally applicable for all hair types, insofar as possible, and mild to the hair and skin.

In order to satisfy the various requirements, a multitude of synthetic polymers were developed as firming active ingredients, which can be used in styling agents. The polymers can be divided into cationic, anionic, nonionic and amphoteric firming polymers. In some cases, the polymers create a polymer film when used on the hair, which lends the hairstyle a strong hold and is sufficiently flexible in order to not break under stress. If the polymer film is too brittle, so-called flakes form, i.e. residue, which detach during movement of the hair and give the impression that the user of the corresponding styling agent has dandruff. Similar problems arise when waxes are used as a firming active ingredient in the styling agent. If the styling agent is a gel or a paste, the polymers may also have thickening properties.

Known anionic polymers that are used in hair fixing products are copolymers with two or more structural units. Certain crosslinked copolymers of this type with the INCI designation acrylates, vinyl neodecanoate crosspolymer and their use in agents for temporary hair deformation are described in the international patent applicants WO 2010/00956 A1 and WO 2016/142011 A1. Such copolymers are available, for example, under the trade name Aculyn® 38 (INCI: acrylates/neodecanoate crosspolymer).

Additional anionic polymers that are used in hair fixing products are crosslinked anionic amphiphilic polymers that contain a (meth) acrylic acid unit and a (meth) acrylic acid oxyalkylene alkyl ester unit. Such polymers are described in the international patent application WO 2016/142013 A1 and are commercially available, for example, under the name BALANCE® RTF (INCI: acrylates/ceteareth-20 methacrylate crosspolymer). In styling products, this polymer has the task of a thickening agent and film former.

### BRIEF SUMMARY

The present disclosure addresses the problem of providing additional polymer combinations, which are exemplified by good film-forming and/or fixing properties, have a very high degree of hold without the need to dispense with flexibility and good moisture resistance—particularly sweat and water resistance—and are also suitable for production of stable viscous and stable transparent cosmetic preparations. In particular, currently available styling agents can be improved, because a good combination of stiffness and long-term hold (high humidity curl retention) is not always sufficiently guaranteed. Therefore, the present disclosure addresses the problem of preparing such styling agents that provide the aforementioned properties, for example with regard to good stiffness and a good, long-term hold.

As contemplated herein, this was achieved by employing a combination of two specific anionic copolymers.

The present disclosure provides:

1. A cosmetic preparation for temporary deformation of keratinous fibers, which contains:

a) at least one crosslinked anionic copolymer (a), which
contains at least one structural unit according to formula (a1)

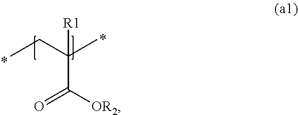

wherein R¹ denotes —H or —CH₃ and R² denotes —H or —CH₃ or —CH₂CH₃ or —CH₂CH₂CH₃ or —CH(CH₃)₂,
and contains at least one additional structural unit according to the formula (a2) differing from the structural unit (a1)

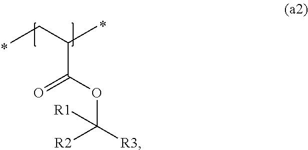

wherein R¹ and R², independently from each other, denote H or —CH₃ or —CH₂CH₃ or —CH₂CH₂CH₃ or —CH(CH₃)₂ and R³ denotes a saturated or unsaturated, straight-chain or branched C₆₋₂₂ hydrocarbon radical and
b) at least one crosslinked acrylate copolymer (b) differing from the copolymer (a), which contains at least one of the following structural units (b1) and (b2):

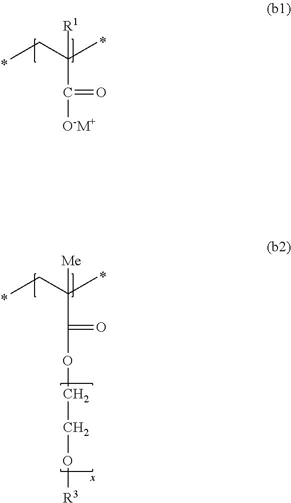

where
R¹ denotes a hydrogen atom or a methyl group,
R³ denotes a (C₈-C₃₀)-alkyl group,
M⁺ denotes a physiologically tolerated cation and
x denotes an integer from 5 to about 35.

2. The cosmetic preparation according to point 1, wherein the copolymer (a) is comprised of at least one of the following monomer units:
at least one (meth)acrylic acid unit,
at least one vinyl neodecanoate unit.

3. The cosmetic preparation according to one of the points above, wherein the copolymer (a) is produced by emulsion polymerization.

4. The cosmetic preparation according to one of the points above, wherein the preparation contains the copolymer (a) in a proportion of from about 0.1 to about 10 wt. %, for example from about 0.5 to about 7.0 wt. %, such as from about 0.7 to about 5.0 wt. %, relative to the total weight of the cosmetic preparation.

5. The cosmetic preparation according to one of the points above, wherein the anionic copolymer (a) includes a copolymer with the INCI designation: acrylates/neodecanoate crosspolymer, such as Aculyn® 38 (Rohm&Haas).

6. The cosmetic preparation according to one of the points above, wherein the radical R¹ in the structural unit (b1) denotes a methyl group.

7. The cosmetic preparation according to one of the points above, wherein R³ in the structural unit (b2) of the crosslinked acrylate copolymer (b) denotes a (C₁₂-C₂₀)-alkyl group, such as a (C₁₄-C₂₀)-alkyl group and in one embodiment a (C₁₆-C₁₈)-alkyl group.

8. The cosmetic preparation according to one of the points above, wherein the x in the structural unit (b2) of the crosslinked acrylate copolymer (b) denotes an integer from about 10 to about 24, such as from about 16 to about 22, for example about 20.

9. The cosmetic preparation according to one of the points above, wherein R³ in the structural unit (b2) of the crosslinked acrylate copolymer (b) denotes a combination of linear C₁₆- and C₁₈-alkyl groups.

10. The cosmetic preparation according to one of the points above, wherein the preparation contains the anionic copolymer (b) in a proportion of from about 0.1 to about 10 wt. %, such as from about 0.5 to about 7.0 wt. %, for example from about 0.7 to about 5.0 wt. %, relative to the total weight of the cosmetic preparation.

11. The cosmetic preparation according to one of the points above, wherein the anionic acrylate copolymer (b) includes a crosspolymer with the INCI designation acrylates/ceteareth-20 methacrylate crosspolymer, such as BALANCE® RCF (AkzoNobel).

12. The cosmetic preparation according to one of the points above, wherein the anionic copolymer (a) includes Aculyn® 38 (Rohm&Haas) and the anionic copolymer (b) includes BALANCE® RCF (AkzoNobel).

13. The cosmetic preparation according to one of the points above, which, relative to the total weight of the cosmetic preparation, contains:

from about 0.1 to about 10 wt. % of the anionic copolymer (a), and
from about 0.1 to about 10 wt. % of the anionic copolymer (b).

14. The cosmetic preparation according to one of the points above, containing, relative to the total weight of the cosmetic preparation,

from about 0.7 to about 5.0 wt. % of the anionic copolymer (a), and
from about 0.7 to about 5.0 wt. % of the anionic copolymer (b).

15. The cosmetic preparation according to one of the points above, wherein the preparation additionally contains at least one copolymer (c) which is different from the copolymers (a) and (b), particularly an anionic or nonionic polymer (c).

16. The cosmetic preparation according to one of the points above, exemplified in that it additionally contains, relative to its total weight,

c) polyvinylpyrrolidone and/or vinylpyrrolidone/vinyl acetate copolymer, and in a particular embodiment polyvinylpyrrolidone.

17. The cosmetic preparation according to point 16, exemplified in that the proportion by weight of the polyvinylpyrrolidone and/or vinylpyrrolidone/vinyl acetate copolymer c) is from about 1.0 to about 10 wt. % of the total weight of the cosmetic preparation, such as from about 2.0 to about 8.5 wt. % and in one embodiment from about 3.0 to about 7.0 wt. %.

18. The cosmetic preparation according to one of the points above, wherein the preparation contains water and the proportion by weight of the water of the cosmetic preparation is from about 50 and about 95 wt. %, such as from about 60 and about 90 wt. % and in one embodiment from about 65 and about 85 wt. %.

19. The cosmetic preparation according to one of the points above, wherein the preparation is provided as hair gel, hairspray, mousse or hair wax, and in a particular embodiment as hair gel.

20. The use of a cosmetic preparation according to one of points 1 to 19 for temporary deformation of keratinous fibers.

21. The use of a cosmetic preparation according to one of points 1 to 19 for improvement of the hold of temporarily deformed keratinous fibers.

22. A method for temporary deformation of keratinous fibers, such as human hair, where the cosmetic preparation according to one of points 1 to 19 is applied on keratinous fibers.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Surprisingly, it was discovered in the context of the present disclosure that a combination of two known components that are already used in styling products can achieve an improved hold of styling products. Other conventionally desirable properties of styling products, such as moisture resistance, stiffness and low stickiness are retained in the process. Such a good combination of properties was not to be expected even when the individual components are known and was surprising. Experiments showed that the combination of the two components produced a strong overadditive, in other words a synergistic effect with regard to the hold, which is manifested in the 3PB test (3-point bending test).

As contemplated herein, the term keratinous fibers includes fur, wool and feathers, and in one embodiment includes human hair.

Particular components of the cosmetic preparation described herein are the crosslinked copolymer (a) and the crosslinked copolymer (b) differing from copolymer (a).

The cosmetic preparation of the present disclosure contains the crosslinked copolymer (a) and crosslinked acrylate copolymer (b) in suitable quantities for a styling agent, which can be adjusted for the particular application and packaging.

The inventive preparation can contain the crosslinked copolymer (a), for example, in a quantity of from about 0.1 to about 10 wt. % relative to the total weight of the disclosed preparation. Proportions of the copolymer (a) from about 0.5 to about 7.0 wt. %, such as from about 0.7 to about 5.0 wt. % may be employed, specified as the solids content of active substance in the cosmetic preparation.

This cosmetic preparation contains the crosslinked acrylate copolymer (b) in a quantity from about 0.1 to about 10 wt. %, such as from about 0.5 to about 7.0 wt. %, and in one embodiment from about 0.7 to about 5.0 wt. % relative to the total weight of the cosmetic preparation, specified as the solids content of active substance in the cosmetic preparation.

The disclosed agents contain a crosslinked copolymer (a) as a first component, which includes at least two structural units of formulae (a1) and (a2). Additional structural units can also be present.

The crosslinked copolymer (a) includes a unit of formula (a1) as a first structural unit

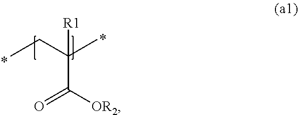

wherein R1 denotes —H or —CH₃ and R2 denotes —H or —CH₃ or —CH₂CH₃ or —CH₂CH₂CH₃ or —CH(CH₃)₂. If R1 denotes —H, the monomers to which the structural unit (a1) can be attributed are acrylic acid or acrylic acid ester; where R1=—CH₃, the monomers to which the structural unit (a1) can be attributed are methacrylic acid or methacrylic acid ester.

Exemplary monomers to which the structural unit (a1) can be attributed are acrylic acid, methacrylic acid, acrylic acid methyl ester, methacrylic acid methyl ester, acrylic acid ethyl ester, methacrylic acid ethyl ester, acrylic acid propyl ester, methacrylic acid propyl ester, acrylic acid isopropyl ester and methacrylic acid isopropyl ester.

Exemplary agents contain acrylic acid or acrylic acid ester as a monomer module in copolymer (a). Such agents are exemplified in that the copolymer (a) contains structural units of formula (a1-1)

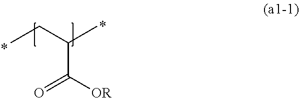

wherein R denotes —H or —CH₃ or —CH₂CH₃ or —CH₂CH₂CH₃ or —CH(CH₃)₂. Such copolymers (a) where the radical R denotes H are exemplary. In one embodiment copolymers (a) have structural units of formula (a1-2).

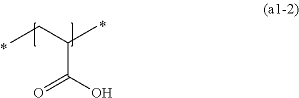

The additional structural unit, which is contained in copolymer (a), can be described by the formula (a2)

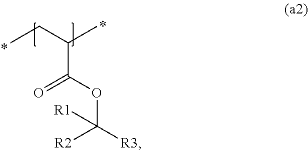

wherein R1 and R2, independently from each other, denote H or —CH₃ or —CH₂CH₃ or —CH₂CH₂CH₃ or —CH(CH₃)₂ and R3 denotes a saturated or unsaturated, straight-chain or branched C₆₋₂₂ hydrocarbon radical.

Exemplary representatives of formula (a2) are structural units where R1=R2=H. Therefore, in one embodiment, structural units (a2) can be described by formula (a2-1)

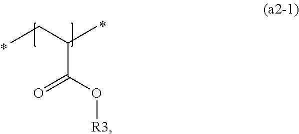

wherein R3 denotes a saturated or unsaturated, branched C₆₋₂₂-hydrocarbon radical, such as a neoheptyl, neooctyl, neononyl or neodecyl radical.

As contemplated herein, agents exemplified in that the copolymer (a) contains structural units of formula (a2-2) are employed in an embodiment.

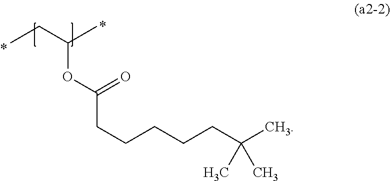

The monomers of formula (I) and (II) may be contained within certain limits in copolymer (a). The agents as contemplated herein are exemplified in that they contain copolymers(s) (a), which contain
from about 10 to about 95 mol. %, such as from about 15 to about 85 mol. % and in one embodiment from about 20 to about 80 mol. % monomers of formula (a1), such as (a1-2) and
from about 5 to about 90 mol. %, such as from about 7.5 to about 80 mol. % and in one embodiment 10 to 60 mol. % monomers of formula (a2), such as (a2-2).

Exemplary crosslinked copolymers (a) are produced by employing polymerization of (meth)acrylic acid, vinyl neodecanoate and, optionally, additional monomers, for example by employing polymerization of acrylic acid, vinyl neodecanoate and, optionally additional monomers.

Exemplary crosslinked copolymers (a) are produced by employing emulsion polymerization of (meth)acrylic acid, vinyl neodecanoate and, optionally, additional monomers, for example by employing emulsion polymerization of acrylic acid, vinyl neodecanoate and, optionally additional monomers.

The molar masses of exemplary crosslinked copolymers (a) are from about 10 to about 750 kDa, such as from v25 to about 500 kDa, for example from about 30 to about 400 kDa, and in one embodiment from about 4 to about 250 kDa.

Suitable crosslinked copolymers (a) are commercially available under the INCI designation acrylates/neodecanoate crosspolymer. The crosslinked copolymer (a) Aculyn® 38 from Rohm & Haas is exemplary thereof. In the commercially available form, this has a solids content of approximately 28 to approximately 33 wt. % and a pH value of from about 2.1 to about 3.2.

An exemplary cosmetic preparation for temporary deformation of keratinous fibers contains:

(a) at least one crosslinked copolymer (a), which is composed of at least one of the following polymer units:
(a1) at least one (meth)acrylic acid unit,
(a2) at least one vinyl neodecanoate unit
and
(b) at least one crosslinked acrylate copolymer (b) differing from the copolymer (a), which includes at least one of the following structural units (b1) and (b2):

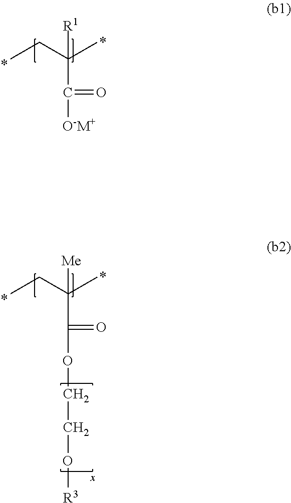

where
R¹ denotes a hydrogen atom or a methyl group,
R³ denotes a (C₈-C₃₀)-alkyl group,
M⁺ denotes a physiologically tolerated cation and
x denotes an integer from 5 to about 35

The crosslinked anionic acrylate copolymer (b) includes at least the structural units (b1) and (b2).

The crosslinked acrylate copolymer (b) is amphiphilic based on the integral structural units. A person skilled in the art understands “amphiphile” to generally mean that one and the same molecule includes hydrophilic structural elements (e.g. of formula (b1)) and lipophilic structural elements (e.g. of formula (b2)).

In the above formulae and all formulae below, a chemical bond bearing the symbol * denotes a free valence of the corresponding structural fragment. The ammonium ion and cationic organic compounds having a quarternized nitrogen atom are exemplary as physiologically compatible cations M⁺ for compensation of the negative charge of the amphiphilic, anionic polymer metal cations of the physiologically compatible metals from the groups Ia, Ib, IIa, IIb, IIIb, VIa or VIII of the periodic table of elements. Cationic organic compounds having a quaternized nitrogen atom are, for example, produced by employing protonation of primary, secondary or tertiary organic amines with an acid, or by employing permanent quaternization of said organic amines. Examples of such cationic organic ammonium compounds are 2-ammonioethanol and 2-trimethylammonioethanol.

The terms “crosslinked” and “crosslinking” in the context of the present disclosure are understood to mean the linking of polymer chains with each other by employing covalent chemical bonding with formation of a network. This covalent linking of polymer chains may take place by employing direct covalent bonding or by employing a molecular fragment bridging the polymer chain. The molecular fragment connects to the polymer chains bridged by the molecular fragment by employing covalent chemical bonding in each case.

The crosslinking of the crosslinked copolymers (a) and (b) can be produced using at least one crosslinking monomer. In the process, it is preferable that the crosslinking monomers are selected from at least one compound of the group including polyunsaturated aromatic monomers (such as divinylbenzene, divinylnaphthalene, trivinylbenzene), polyunsaturated alicyclic monomers (such as 1,2,4-trivinylcyclohexane), di-functional esters of phthalic acid (such as diallyl phthalate), polyunsaturated aliphatic monomers (such as dienes, trienes, tetraenes such as isoprene, 1,3-butadiene, 1,5-hexadiene, 1,5,9-decatriene, 1,9-decadiene, 1,5-heptadiene), polyalkenyl ethers (such as triallyl pentaerythritol, diallyl pentaerythritol, diallyl sucrose, octaallyl sucrose, trimethylolpropane diallyl ether), polyunsaturated esters of polyalcohols or polyacids (such as 1,6-hexanediol di(meth)acrylate, tetramethylene tri(meth)acrylate, allyl acrylate, diallyl itaconate, diallyl fumarate, diallyl maleate, trimethylolpropane tri(meth)acrylate, trimethylolpropane di(meth)acrylate, polyethylene glycol di(meth)acrylate), alkylene bisacrylamides (such as methylenebisacrylamide, propylene bisacrylamide) hydroxy- and carboxy derivatives of methylene bisacrylamide (such as N,N′-bis-methylol methylene-bisacrylamide), polyethylene glycol di(meth)acrylates (such as ethylene glycol di(meth)acrylate, diethylene glycol di(meth)acrylate, triethylene glycol di(meth)acrylate), polyunsaturated silanes (such as dimethylvinylsilane, methyltrivinylsilane, allyl dimethylvinylsilane, diallyl dimethylsilane, tetravinylsilane), n-methylolacrylamide; n-alkoxy(meth) acrylamide, wherein the alkoxy group is a (C1 to C18)-alkoxy group, unsaturated hydrolyzable silanes (such as triethoxyvinylsilane, tris-isopropoxy-vinylsilane, 3-triethoxysilyl-propylmethacrylate), hydrolyzable silanes (such as ethyltriethoxysilane, ethyltrimethoxysilane), epoxy-substituted hydrolyzable silanes (such as 2-(3,4-epoxycyclohexyl) ethyltriethoxysilane, 3-glycidoxypropyl trimethyoxysilane), polyisocyanates (such as 1,4-diisocyanatobutane, 1,6-diisocyanatohexane, 1,4-phenylenediamine diisocyanate, 4,4′-oxybis(phenylisocyanate)), unsaturated epoxides (such as glycidylmethacrylates, allyl glycidyl ether), polyepoxides (such as diglycidyl ether, 1,2,5,6-diepoxy hexane, ethylene glycol diglycidyl ether), ethoxylated polyols (such as diols, triols and diphenols, each ethoxylated with about 2 to about 100 moles of ethylene oxide per mole of hydroxyl groups and terminated with a polymerizable unsaturated group, such as, vinyl ether, allyl ether, acrylate ester, methacrylate ester; examples include bisphenol A ethoxylated di(meth)acrylate, bisphenol F ethoxylated di(meth)acrylate, ethoxylated trimethylolpropane tri(meth)acrylates, acrylate and methacrylate esters of polyols having at least two acrylate ester or methacrylate ester-functionalities (such as trimethylolpropane triacrylate (TMPTA), trimethylolpropane ethoxylated (15) triacrylate (TMPEO15TA), trimethylolpropane dimethacrylate, triethyleneglycoldimethacrylate (TEGDMA), with 30 mol of ethylene oxide ethoxylated bisphenol A-dimethacrylate (EOBDMA)).

As contemplated herein, the copolymer (b) can be composed of additional structure units. In a preferred embodiment, the copolymer (b) includes at least about 30 wt. %, such as from about 40 to about 98 wt. % and in one embodiment at least about 50 to about 95 wt. % of monomers (b1) and (b2). However, in an exemplary embodiment of the present disclosure, the copolymer (b) includes only units (b1) and (b2) and units to be crosslinked, i.e. it is composed of these structural units.

The at least one unit (b1) is a (meth)acrylic acid unit and, as contemplated herein, can be a methacrylic acid unit and/or acrylic acid unit. In one embodiment, the unit (b1) is a methacrylic acid unit (R¹ in formula (b1) denotes a methyl group). Corresponding acrylic polymers (b) have been found to be suitable for cosmetic applications.

In one embodiment, x in the structural unit (b2) of the crosslinked acrylate copolymer (b) denotes an integer from about 10 to about 24, preferably from about 16 to about 22, most preferably about 20.

In one embodiment, R³ in unit (b2) of the crosslinked acrylate copolymer (b) denotes a (C₁₂-C₂₀)-alkyl group, such as a (C₁₄-C₂₀)-alkyl group, for example a (C₁₆-C₁₈)-alkyl group. The alkyl group in this context can be linear, but can also be branched. R³ denotes, for example, a combination of linear C₁₆- and C₁₈-alkyl groups, i.e. stearyl- and cetyl groups (INCI: ceteareth). Corresponding acrylic polymers (b) have been found to be suitable for cosmetic applications.

In one embodiment, crosslinked acrylate copolymer (b) includes a crosslinked acrylate copolymer with the INCI designation acrylates-ceteareth-20 methacrylate crosspolymer. In one embodiment, the crosslinked acrylate copolymer (b) includes a crosslinked acrylate copolymer available under the trade name BALANCE® RCF (AkzoNobel). The latter is an approximately 30 wt. % dispersion in water.

Additional exemplary crosslinked acrylate copolymers (b) are identified by the INCI designation acrylates/steareth-20 methacrylate crosspolymer. In such crosslinked acrylate copolymers, the at least one unit (a1) is a (meth)acrylic acid unit and, as contemplated herein, can be a methacrylic acid unit and/or acrylic acid unit. They have 20 units of ethylene oxide and are etherified with stearyl alcohol. One such polymer, for example, is available under the trade name Aculyn® 88 (Rohm & Haas). In the commercially available form, this has a solids content of approximately 28 to approximately 33 wt. % and a pH value of from about 3.3 to about 4.3.

Compared to alternative cosmetic agents, the cosmetic preparations as contemplated herein are exemplified by an improved long-term hold, in addition to the aforementioned advantages. A weight ratio of polymers a) and b) in the cosmetic preparation of from about 5:1 to about 1:5, such as from about 3:1 to about 1:3 and in one embodiment from about 2:1 to about 1:2 has been found to be suitable for the cosmetic properties of the disclosed agent.

In an exemplary embodiment of the present disclosure, the cosmetic preparation contains the copolymer commercially available under the name Aculyn® 38 as the crosslinked copolymer (a) and the copolymer commercially available under the name BALANCE® RCF as the crosslinked acrylate copolymer (b). Good results with regard to a combination of stiffness and long-term hold were achieved with this combination. This polymer combination is suitable for styling products in gel form.

Additional properties generally desirable in styling products, such as moisture resistance and low stickiness, are also provided with this combination, for example when packaged as hair gel.

The crosslinked copolymers (a) and (b) may be used in partially neutralized or neutralized form in the cosmetic preparation. At least one alkanolamine may be used for neutralization. The alkanolamines used as an alkalization agent in the context of the present disclosure may be selected from primary amines with a C₂-C₆-alkyl base body having at least one hydroxyl group. In some embodiments, alkanolamines are selected from the group of 2-aminoethan-1-ol (monoethanolamine), 3-(2-hydroxyethyl)-amine (triethanolamine), 3-aminopropan-1-ol, 4-amino-butan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropan-1,2-diol, 2-amino-2-methylpropan-1,3-diol. As contemplated herein, alkanolamines may be selected from the group of 2-aminoethan-1-ol, 2-amino-2-methylpropan-1-ol and 2-amino-2-methyl-propan-1,3-diol. In this context, 2-amino-2-methylpropanol has been found to be suitable as a neutralization agent. Therefore, exemplary agents as contemplated herein contain 2-amino-2-methylpropanol. The 2-amino-2-methylpropanol may be used in the disclosed agents in a quantity which, in some embodiments, does not exceed the quantity required for neutralization of copolymers (a) and (b). For example, the quantities of 2-amino-2-methylpropanol used in the disclosed preparations are from about 80 to about 100%, such as from about 90 to about 100% and in one embodiment from about 95 to about 100% of the quantity required for complete neutralization of copolymers (a) and (b). In a preferred embodiment, the proportion by weight of the 2-amino-2-methylpropanol to the total weight of the cosmetic agent is from about 0.05 to about 7.0 wt. %, such as from about 0.1 to about 5.0 wt. % and in one embodiment from about 0.1 to about 3.0 wt. %.

In summary, an exemplary cosmetic preparation for temporary deformation of keratinous fibers contains, relative to its total weight:

(a) from about 0.7 to about 5.0 wt. % of at least one crosslinked copolymer (a) which is at least one of the following monomer units:
at least one (meth)acrylic acid unit,
at least one vinyl neodecanoate unit
   and
   (b) from about 0.7 to about 5.0 wt. % of at least one crosslinked acrylate copolymer (b) differing from the copolymer (a), which includes at least one of the following structural units (b1) and (b2):

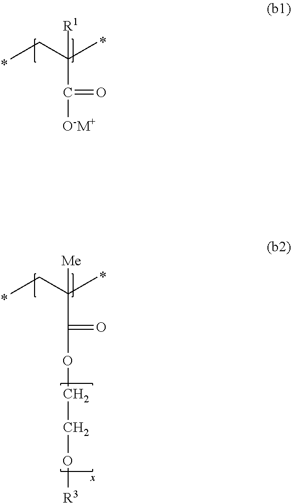

where
R¹ denotes a methyl group,
R³ denotes a (C₁₄-C₂₀)-alkyl group,
M⁺ denotes a physiologically tolerated cation and
x denotes an integer from about 16 to about 22

The cosmetic preparation according to the present disclosure may contain one or more additional component(s) which differ(s) from the copolymers (a) and (b) and aid the thickening agent or the gel formation or film formation. Examples are cationic, anionic, nonionic or amphoteric polymers. The proportion by weight of these additional components to the total weight of the cosmetic preparation can be comparatively low due to the presence of components (a) and (b) and, for example, can be from about 0.02 to about 3 wt. %, such as from about 0.05 to about 1.5 wt. % and in one embodiment from about 0.2 to about 0.8 wt. %.

Examples are acrylamide/ammonium acrylate copolymer, acrylamides/DMAPA acrylates/methoxy PEG methacrylate copolymer, acrylamidopropyltrimonium chloride/acrylamide copolymer, acrylamidopropyltrimonium chloride/acrylates copolymer, acrylates/acetoacetoxyethyl methacrylate copolymer, acrylates/acrylamide copolymer, acrylates/ammonium methacrylate copolymer, acrylates/t-butylacrylamide copolymer, acrylates/C1-2 succinates/hydroxyacrylates copolymer, acrylates/lauryl acrylate/stearyl acrylate/ethylamine oxide methacrylate copolymer, acrylates/octylacrylamide copolymer, acrylates/octylacrylamide/diphenyl amodimethicone copolymer, acrylates/stearyl acrylate/ethylamine oxide methacrylate copolymer, acrylates/VA copolymer, acrylates/VP copolymer, adipic acid/diethylenetriamine copolymer, adipic acid/dimethylaminohydroxypropyldiethylenetriamine copolymer, adipic acid/epoxypropyl diethylenetriamine copolymer, adipic acid/isophthalic acid/neopentyl glycol/trimethylolpropane copolymer, allyl stearate/VA copolymer, aminoethylacrylate phosphate/acrylates copolymer, aminoethylpropanediol-acrylates/acrylamide copolymer, aminoethylproanediol-AMPD-acrylates/diacetoneacrylamide copolymer, ammonium VA/acrylates copolymer, AMPD-acrylates/diacetoneacrylamide copolymer, AMP-acrylates/allyl methacrylate copolymer, AMP-acrylates/C1-18 alkyl acrylates/C1-8 alkyl acrylamide copolymer, AMP-acrylates/diacetoneacrylamide copolymer, AMP-acrylates/dimethylaminoethylmethacrylate copolymer, bacillus/rice bran extract/soybean extract ferment filtrate, bis-butyloxyamodimethicone/PEG-60 copolymer, butyl acrylate/ethylhexyl methacrylate copolymer, butyl acrylate/hydroxypropyl dimethicone acrylate copolymer, butylated PVP, butyl ester of ethylene/MA copolymer, butyl ester of PVM/MA copolymer, calcium/sodium PVM/MA copolymer, corn starch/acrylamide/sodium acrylate copolymer, diethylene glycolamine/epichlorohydrin/piperazine copolymer, dimethicone crosspolymer, diphenyl amodimethicone, ethyl ester of PVM/MA copolymer, hydrolyzed wheat protein/PVP crosspolymer, isobutylene/ethylmaleimide/hydroxyethylmaleimide copolymer, isobutylene/MA copolymer, isobutylmethacrylate/bis-hydroxypropyl dimethicone acrylate copolymer, isopropyl ester of PVM/MA copolymer, lauryl acrylate crosspolymer, lauryl methacrylate/glycol dimethacrylate crosspolymer, MEA-sulfite, methacrylic acid/sodium acrylamidomethyl propane sulfonate copolymer, methacryloyl ethyl betaine/acrylates copolymer, octylacrylamide/acrylates/butylaminoethyl methacrylate copolymer, PEG/PPG-25/25 dimethicone/acrylates copolymer, PEG-8/SMDI copolymer, polyacrylamide, polyacrylate-6, polybeta-alanine/glutaric acid crosspolymer, polybutylene terephthalate, polyester-1, polyethylacrylate, polyethylene terephthalate, polymethacryloyl ethyl betaine, polypentaerythrityl terephthalate, polyperfluoroperhydrophenanthrene, polyquaternium-1, polyquaternium-2, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-22, polyquaternium-24, polyquaternium-27, polyquaternium-28, polyquaternium-29, polyquaternium-30, polyquaternium-31, polyquaternium-32, polyquaternium-33, polyquaternium-34, polyquaternium-35, polyquaternium-36, polyquaternium-37, polyquaternium-39, polyquaternium-45, polyquaternium-46, polyquaternium-47, polyquaternium-48, polyquaternium-49, polyquaternium-50, polyquaternium-55, polyquaternium-56, polysilicone-9, polyurethane-1, polyurethane-6, polyurethane-10, polyvinyl acetate, polyvinyl butyral, polyvinylcaprolactam, polyvinylformamide, polyvinyl imidazolinium acetate, polyvinyl methyl ether, potassium butyl ester of PVM/MA copolymer, potassium ethyl ester of PVM/MA copolymer, PPG-70 polyglyceryl-10 ether, PPG-12/SMDI copolymer, PPG-51/SMDI copolymer, PPG-10 sorbitol, PVM/MA copolymer, PVP, PVP/VA/itaconic acid copolymer, PVP/VA/vinyl propionate copolymer, rhizobian gum, rosin acrylate, shellac, sodium butyl ester of PVM/MA copolymer, sodium ethyl ester of PVM/MA copolymer, sodium polyacrylate, sterculia urens gum, terephthalic acid/isophthalic acid/sodium isophthalic acid sulfonate/glycol copolymer, trimethylolpropane triacrylate, trimethylsiloxysilylcarbamoyl pullulan, VA/crotonates copolymer, VA/crotonates/methacryloxybenzophenone-1 copolymer, VA/crotonates/vinyl neodecanoate copolymer, VA/crotonates/vinyl propionate copolymer, VA/DBM copolymer, VA/vinyl butyl benzoate/crotonates copolymer, vinylamine/vinyl alcohol copolymer, vinyl caprolactam/VP/dimethylaminoethyl methacrylate copolymer, VP/acrylates/lauryl methacrylate copolymer, VP/dimethylaminoethylmethacrylate copolymer, VP/DMAPA acrylates copolymer, VP/hexadecene copolymer, VP/VA copolymer, VP/vinyl caprolactam/DMAPA acrylates copolymer, yeast palmitate and styrene/VP copolymer.

The additional component acting as a gel former is for example a homopolyacrylic acid (INCI: carbomer), which is commercially available under the name Carbopol® in different embodiments. The carbomer is preferably contained in a proportion of from about 0.02 to about 3 wt. %, such as from about 0.05 to about 1.5 wt. % and in one embodiment from v 0.2 to about 0.8 wt. %, relative to the total weight of the cosmetic preparation.

To further improve the cosmetic effect, exemplary preparations contain, in addition to the crosslinked copolymers (a) and (b) and an optionally added thickening agent or gel former, a film-forming polymer (c) differing from these ingredients, such as an anionic or nonionic polymer (c).

Examples of nonionic polymers are:

vinylpyrrolidone/vinylester copolymers, which are sold, for example, under the trade name Luviskol (BASF). Luviskol VA 64 and Luviskol VA 73, in each case vinylpyrrolidone/vinylacetate-copolymers, are exemplary nonionic polymers.

cellulose ethers, such as hydroxypropyl cellulose, hydroxyethyl cellulose and methylhydroxypropyl cellulose, are sold, for example, under the trade name Culminaland Benecel (AQUALON).

shellac.
polyvinylpyrrolidone, which is sold, for example, under the name Luviskol (BASF).
siloxanes. These siloxanes can be water-soluble or non-water-soluble. Fluid and non-fluid siloxanes are suitable, wherein non-fluid siloxanes are to be understood to mean such compounds having a boiling point above 200° C. under normal pressure. Exemplary siloxanes are polydialkylsiloxanes, such as polydimethylsiloxane, polyalkylarylsiloxanes, such as polyphenylmethylsiloxane, ethoxylated polydialkylsiloxanes and polydialkylsiloxanes which contain amine and/or hydroxyl groups.
glycosidically substituted silicones.

Due to their cosmetic effect in combination with the copolymers a) and b), as contemplated herein, exemplary film-forming polymers are polyvinylpyrrolidones (INCI designation: PVP) and vinylpyrrolidone/vinylacetate copolymers (INCI designation VP/VA copolymer). The proportion by weight of these polymers may be limited to quantities between about 1.0 and about 10 wt. %. Suitable cosmetic preparations as contemplated herein, therefore, are exemplified in that they contain an additional from about 1.0 to about 10 wt. % of polyvinylpyrrolidone and/or vinylpyrrolidone/vinylacetate-copolymer, in a particular embodiment polyvinylpyrrolidone, relative to their total weight. Exemplary cosmetic agents have a proportion by weight of from about 2.0 to about 8.5 wt. %, such as from about 3.0 to about 7.0 wt. % of polyvinylpyrrolidone and/or vinylpyrrolidone/vinylacetate-copolymers c), relative to the total weight of the cosmetic agent.

The disclosed cosmetic preparation can contain additional ingredients of styling products. Additional care substances, in particular, can be mentioned as additional suitable auxiliary substances and additives. For example, the agent can contain at least one protein hydrolyzate and/or one of its derivatives as a care substance. Protein hydrolyzates are product mixtures produced with acidic, basic or enzymatically catalyzed decomposition of proteins. The term protein hydrolyzates in the context of the present disclosure is understood to also mean total hydrolyzates, as well as individual amino acids and derivatives thereof, as well as mixtures of various amino acids. The mol weight of the protein hydrolyzates to be used as contemplated herein is between about 75, the mol weight for glycine, and about 200,000, such as from about 75 to about 50,000 and in one embodiment from about 75 to about 20,000 Daltons.

The disclosed agent can also contain at least one vitamin, a provitamin, a vitamin precursor and/or a derivative thereof as a care substance. As contemplated herein, such vitamins, provitamins and vitamin precursors which are classified in the groups A, B, C, E, F and H are suitable.

Similar to the addition of glycerin and/or propylene glycol, the addition of panthenol increases the flexibility of the polymer film formed using the inventive agent.

The disclosed agents can also contain at least one plat extract, as well as mono- and/or oligosaccharides and/or lipids as care substances.

Furthermore, oil bodies are suitable as care substances. The natural and synthetic cosmetic oil bodies include, for example, vegetable oils, liquid paraffin oils, isoparaffin oils and synthetic hydrocarbons, and di-n-alkyl ethers having a total of between about 12 to about 36 carbon atoms, such as from about 12 to about 24 carbon atoms. Cosmetic agents as contemplated herein contain at least one oil body, such as at least one oil body from the group of silicone oils. The group of silicone oils also includes, for example, dimethicones, which also include cyclomethicones, amino-functional silicones and dimethiconols. Dimethicones can be linear or branched, as well as cyclical or cyclical and branched. Suitable silicone oils or silicone gums are, for example dialkyl- and alkylarylsiloxanes, such as dimethylpolysiloxane and methylphenylpolysiloxane, and alkoxylated, quaternized or anionic derivatives thereof. Cyclical and linear polydialkylsiloxanes, alkoxylated and/or aminated derivatives thereof, dihydroxypolydimethylsiloxanes and polyphenylalkylsiloxanes are used in an embodiment.

Ester oils, i.e. esters of C6-C30 fatty acids with C2-C30 fatty alcohols, for example monoesters of fatty acids with alcohols having from about 2 to about 24 carbon atoms, such as isopropyl myristate (Rilanit® IPM), isononanoic acid C16-18 alkyl esters (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate)(Cetiol®, lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V) are additional suitable care oil bodies.

Furthermore, dicarboxylic acid esters, symmetrical, asymmetrical or cyclic esters of carbonic acid with fatty alcohols, trifatty acid esters of saturated and/or unsaturated linear and/or branched fatty acids with glycerol or fatty acid partial glycerides, which are to be understood as monoglycerides, diglycerides and technical mixtures thereof are suitable as care sub stances.

Furthermore, emulsifiers and/or surfactants may be contained in the disclosed preparation. PEG derivatives of hydrated castor oil are exemplary, which are available, for example, under the name PEG Hydrogenated Castor Oil, such as PEG-30 Hydrogenated Castor Oil, PEG-33 Hydrogenated Castor Oil, PEG-35 Hydrogenated Castor Oil, PEG-36 Hydrogenated Castor Oil or PEG-40 Hydrogenated Castor Oil. As contemplated herein, use of PEG-40 Hydrogenated Castor Oil is exemplary. These may be contained in a quantity of from about 0.05 to about 1.5 wt. %, such as from about 0.1 to about 1.0 wt. %, and in embodiments from about 0.2 to about 0.8 wt. % or from about 0.3 to about 0.6 wt. %.

The disclosed cosmetic agents contain the ingredients and/or active ingredients in a cosmetically acceptable carrier.

Exemplary cosmetically acceptable carriers are aqueous, alcoholic or aqueous-alcoholic media preferably having at least 10 wt. % water, relative to the total weight of the agent.

In one embodiment, the inventive cosmetic carrier contains water, such as in a quantity that is at least about 10 wt. %, such as at least about 20.0 wt %, for example at least about 40 wt. % water relative to the total weight of the agent. Exemplary cosmetic agents have a proportion of water from about 50 to about 95 wt. %, such as from about 60 to about 90 wt. % and in one embodiment from about 65 to about 85 wt. %, relative to their total weight.

Low alcohols having 1 to 4 carbon atoms used for cosmetic purposes, such as ethanol and isopropanol, can be used, for example as alcohols.

Examples of water-soluble solvents as a cosolvent are glycerol and/or ethylene glycol and/or 1,2-propylene glycol in a quantity of from about 0 to about 30 wt. % relative to the total agent.

Tabular Overview

A summary of some exemplary cosmetic agents is provided in the following tables (specifications in wt. % relative to the total weight of the cosmetic agent, unless otherwise specified).

| | Formula | Formula | Formula | Formula | Formula |
|---|---|---|---|---|---|
| | 1 | 2 | 3 | 4 | 5 |
| Copolymer a) | 0.1 to 10 | 0.2 to 8.0 | 0.5 to 7.0 | 0.6 to 4.0 | 0.7 to 5.0 |
| Copolymer b) | 0.1 to 10 | 0.2 to 8.0 | 0.5 to 7.0 | 0.6 to 4.0 | 0.7 to 5.0 |
| Misc | add 100 | add 100 | add 100 | add 100 | add 100 |

| | Formula | Formula | Formula | Formula | Formula |
|---|---|---|---|---|---|
| | 1a | 2a | 3a | 4a | 5a |
| Acrylates/Neodecanoate | 0.1 to 10 | 0.2 to 8.0 | 0.5 to 7.0 | 0.6 to 4.0 | 0.7 to 5.0 |
| Crosspolymer | | | | | |
| Acrylates/Ceteareth-20 | 0.1 to 10 | 0.2 to 8.0 | 0.5 to 7.0 | 0.6 to 4.0 | 0.7 to 5.0 |
| Methacrylate Crosspolymer | | | | | |
| Misc | add 100 | add 100 | add 100 | add 100 | add 100 |

| | Formula | Formula | Formula | Formula | Formula |
|---|---|---|---|---|---|
| | 1b | 2b | 3b | 4b | 5b |
| Aculyn ® 38 | 0.1 to 10 | 0.2 to 8.0 | 0.5 to 7.0 | 0.6 to 4.0 | 0.7 to 5.0 |
| (specifications as | | | | | |
| solids content) | | | | | |
| BALANCE ® RCF | 0.1 to 10 | 0.2 to 8.0 | 0.5 to 7.0 | 0.6 to 4.0 | 0.7 to 5.0 |
| (specifications as | | | | | |
| solids content) | | | | | |
| Misc | add 100 | add 100 | add 100 | add 100 | add 100 |

| | Formula | Formula | Formula | Formula | Formula |
|---|---|---|---|---|---|
| | 6 | 7 | 8 | 9 | 10 |
| Copolymer a) | 0.1 to 10 | 0.2 to 8.0 | 0.5 to 7.0 | 0.6 to 4.0 | 0.7 to 5.0 |
| Copolymer b) | 0.1 to 10 | 0.2 to 8.0 | 0.5 to 7.0 | 0.6 to 4.0 | 0.7 to 5.0 |
| Polyvinylpyrrolidone | 1.0 to 10 | 2.0 to 8.5 | 2.0 to 8.5 | 3.0 to 7.0 | 3.0 to 7.0 |
| Misc | add 100 | add 100 | add 100 | add 100 | add 100 |

| | Formula | Formula | Formula | Formula | Formula |
|---|---|---|---|---|---|
| | 6a | 7a | 8a | 9a | 10a |
| Acrylates/Neodecanoate | 0.1 to 10 | 0.2 to 8.0 | 0.5 to 7.0 | 0.6 to 4.0 | 0.7 to 5.0 |
| Crosspolymer | | | | | |
| Acrylates/Ceteareth-20 | 0.1 to 10 | 0.2 to 8.0 | 0.5 to 7.0 | 0.6 to 4.0 | 0.7 to 5.0 |
| Methacrylate Crosspolymer | | | | | |
| Polyvinylpyrrolidone | 1.0 to 10 | 2.0 to 8.5 | 2.0 to 8.5 | 3.0 to 7.0 | 3.0 to 7.0 |
| Misc | add 100 | add 100 | add 100 | add 100 | add 100 |

| | Formula | Formula | Formula | Formula | Formula |
|---|---|---|---|---|---|
| | 6b | 7b | 8b | 9b | 10b |
| Aculyn ® 38 | 0.1 to 10 | 0.2 to 8.0 | 0.5 to 7.0 | 0.6 to 4.0 | 0.7 to 5.0 |
| (specifications as | | | | | |
| solids content) | | | | | |
| BALANCE ® RCF | 0.1 to 10 | 0.2 to 8.0 | 0.5 to 7.0 | 0.6 to 4.0 | 0.7 to 5.0 |
| (specifications as | | | | | |
| solids content) | | | | | |
| Polyvinylpyrrolidone | 1.0 to 10 | 2.0 to 8.5 | 2.0 to 8.5 | 3.0 to 7.0 | 3.0 to 7.0 |
| Misc | add 100 | add 100 | add 100 | add 100 | add 100 |

| | Formula | Formula | Formula | Formula | Formula |
|---|---|---|---|---|---|
| | 11 | 12 | 13 | 14 | 15 |
| Copolymer a) | 0.1 to 10 | 0.2 to 8.0 | 0.5 to 7.0 | 0.6 to 4.0 | 0.7 to 5.0 |
| Copolymer b) | 0.1 to 10 | 0.2 to 8.0 | 0.5 to 7.0 | 0.6 to 4.0 | 0.7 to 5.0 |
| Vinylpyrrolidone/ | 1.0 to 10 | 2.0 to 8.5 | 2.0 to 8.5 | 3.0 to 7.0 | 3.0 to 7.0 |
| Vinylacetate-Copolymer | | | | | |
| Misc | add 100 | add 100 | add 100 | add 100 | add 100 |

| | Formula | Formula | Formula | Formula | Formula |
|---|---|---|---|---|---|
| | 11a | 12a | 13a | 14a | 15a |
| Acrylates/Neodecanoate | 0.1 to 10 | 0.2 to 8.0 | 0.5 to 7.0 | 0.6 to 4.0 | 0.7 to 5.0 |
| Crosspolymer | | | | | |
| Acrylates/Ceteareth-20 | 0.1 to 10 | 0.2 to 8.0 | 0.5 to 7.0 | 0.6 to 4.0 | 0.7 to 5.0 |
| Methacrylate Crosspolymer | | | | | |
| Vinylpyrrolidone/ | 1.0 to 10 | 2.0 to 8.5 | 2.0 to 8.5 | 3.0 to 7.0 | 3.0 to 7.0 |
| Vinylacetate-Copolymer | | | | | |
| Misc | add 100 | add 100 | add 100 | add 100 | add 100 |

| | Formula | Formula | Formula | Formula | Formula |
|---|---|---|---|---|---|
| | 11b | 12b | 13b | 14b | 15b |
| Aculyn ® 38 | 0.1 to 10 | 0.2 to 8.0 | 0.5 to 7.0 | 0.6 to 4.0 | 0.7 to 5.0 |
| (specifications as | | | | | |
| solids content) | | | | | |
| BALANCE ® RCF | 0.1 to 10 | 0.2 to 8.0 | 0.5 to 7.0 | 0.6 to 4.0 | 0.7 to 5.0 |
| (specifications as | | | | | |
| solids content) | | | | | |
| Vinylpyrrolidone/ | 1.0 to 10 | 2.0 to 8.5 | 2.0 to 8.5 | 3.0 to 7.0 | 3.0 to 7.0 |
| Vinylacetate-Copolymer | | | | | |
| Misc | add 100 | add 100 | add 100 | add 100 | add 100 |

| | Formula | Formula | Formula | Formula | Formula |
|---|---|---|---|---|---|
| | 16 | 17 | 18 | 19 | 20 |
| Copolymer a) | 0.1 to 10 | 0.2 to 8.0 | 0.5 to 7.0 | 0.6 to 4.0 | 0.7 to 5.0 |
| Copolymer b) | 0.1 to 10 | 0.2 to 8.0 | 0.5 to 7.0 | 0.6 to 4.0 | 0.7 to 5.0 |
| Carbomer | 0.02 to 3.0 | 0.05 to 2.0 | 0.05 to 1.5 | 0.2 to 1.0 | 0.4 to 0.8 |
| Misc | add 100 | add 100 | add 100 | add 100 | add 100 |

| | Formula | Formula | Formula | Formula | Formula |
|---|---|---|---|---|---|
| | 16a | 17a | 18a | 19a | 20a |
| Acrylates/Neodecanoate | 0.1 to 10 | 0.2 to 8.0 | 0.5 to 7.0 | 0.6 to 4.0 | 0.7 to 5.0 |
| Crosspolymer | | | | | |
| Acrylates/Ceteareth-20 | 0.1 to 10 | 0.2 to 8.0 | 0.5 to 7.0 | 0.6 to 4.0 | 0.7 to 5.0 |
| Methacrylate Crosspolymer | | | | | |
| Carbomer | 0.02 to 3.0 | 0.05 to 2.0 | 0.05 to 1.5 | 0.2 to 1.0 | 0.4 to 0.8 |
| Misc | add 100 | add 100 | add 100 | add 100 | add 100 |

| | Formula | Formula | Formula | Formula | Formula |
|---|---|---|---|---|---|
| | 16b | 17b | 18b | 19b | 20b |
| Aculyn ® 38 | 0.1 to 10 | 0.2 to 8.0 | 0.5 to 7.0 | 0.6 to 4.0 | 0.7 to 5.0 |
| (specifications as | | | | | |
| solids content) | | | | | |
| BALANCE ® RCF | 0.1 to 10 | 0.2 to 8.0 | 0.5 to 7.0 | 0.6 to 4.0 | 0.7 to 5.0 |
| (specifications as | | | | | |
| solids content) | | | | | |
| Carbomer | 0.02 to 3.0 | 0.05 to 2.0 | 0.05 to 1.5 | 0.2 to 1.0 | 0.4 to 0.8 |
| Misc | add 100 | add 100 | add 100 | add 100 | add 100 |

| | Formula | Formula | Formula | Formula | Formula |
|---|---|---|---|---|---|
| | 21 | 22 | 23 | 24 | 25 |
| Copolymer a) | 0.1 to 10 | 0.2 to 8.0 | 0.5 to 7.0 | 0.6 to 4.0 | 0.7 to 5.0 |
| Copolymer b) | 0.1 to 10 | 0.2 to 8.0 | 0.5 to 7.0 | 0.6 to 4.0 | 0.7 to 5.0 |
| PEG-40 Hydrogenated | 0.05 to 1.5 | 0.1 to 1.0 | 0.2 to 0.9 | 0.3 to 0.8 | 0.4 to 0.6 |
| Castor Oil | | | | | |
| Misc | add 100 | add 100 | add 100 | add 100 | add 100 |

| | Formula | Formula | Formula | Formula | Formula |
|---|---|---|---|---|---|
| | 21a | 22a | 23a | 24a | 25a |
| Acrylates/Neodecanoate | 0.1 to 10 | 0.2 to 8.0 | 0.5 to 7.0 | 0.6 to 4.0 | 0.7 to 5.0 |
| Crosspolymer | | | | | |
| Acrylates/Ceteareth-20 | 0.1 to 10 | 0.2 to 8.0 | 0.5 to 7.0 | 0.6 to 4.0 | 0.7 to 5.0 |
| Methacrylate Crosspolymer | | | | | |
| PEG-40 Hydrogenated | 0.05 to 1.5 | 0.1 to 1.0 | 0.2 to 0.9 | 0.3 to 0.8 | 0.4 to 0.6 |
| Castor Oil | | | | | |
| Misc | add 100 | add 100 | add 100 | add 100 | add 100 |

| | Formula | Formula | Formula | Formula | Formula |
|---|---|---|---|---|---|
| | 21b | 22b | 23b | 24b | 25b |
| Aculyn ® 38 | 0.1 to 10 | 0.2 to 8.0 | 0.5 to 7.0 | 0.6 to 4.0 | 0.7 to 5.0 |
| (specifications as | | | | | |
| solids content) | | | | | |
| BALANCE ® RCF | 0.1 to 10 | 0.2 to 8.0 | 0.5 to 7.0 | 0.6 to 4.0 | 0.7 to 5.0 |
| (specifications as | | | | | |
| solids content) | | | | | |
| PEG-40 Hydrogenated | 0.05 to 1.5 | 0.1 to 1.0 | 0.2 to 0.9 | 0.3 to 0.8 | 0.4 to 0.6 |
| Castor Oil | | | | | |
| Misc | add 100 | add 100 | add 100 | add 100 | add 100 |

| | Formula | Formula | Formula | Formula | Formula |
|---|---|---|---|---|---|
| | 26 | 27 | 28 | 29 | 30 |
| Copolymer a) | 0.1 to 10 | 0.2 to 8.0 | 0.5 to 7.0 | 0.6 to 4.0 | 0.7 to 5.0 |
| Copolymer b) | 0.1 to 10 | 0.2 to 8.0 | 0.5 to 7.0 | 0.6 to 4.0 | 0.7 to 5.0 |
| Water | 50 to 95 | 50 to 95 | 60 to 90 | 60 to 90 | 65 to 85 |
| Misc | add 100 | add 100 | add 100 | add 100 | add 100 |

| | Formula | Formula | Formula | Formula | Formula |
|---|---|---|---|---|---|
| | 26a | 27a | 28a | 29a | 30a |
| Acrylates/Neodecanoate | 0.1 to 10 | 0.2 to 8.0 | 0.5 to 7.0 | 0.6 to 4.0 | 0.7 to 5.0 |
| Crosspolymer | | | | | |
| Acrylates/Ceteareth-20 | 0.1 to 10 | 0.2 to 8.0 | 0.5 to 7.0 | 0.6 to 4.0 | 0.7 to 5.0 |
| Methacrylate Crosspolymer | | | | | |
| Water | 50 to 95 | 50 to 95 | 60 to 90 | 60 to 90 | 65 to 85 |
| Misc | add 100 | add 100 | add 100 | add 100 | add 100 |

| | Formula | Formula | Formula | Formula | Formula |
|---|---|---|---|---|---|
| | 26b | 27b | 28b | 29b | 30b |
| Aculyn ® 38 | 0.1 to 10 | 0.2 to 8.0 | 0.5 to 7.0 | 0.6 to 4.0 | 0.7 to 5.0 |
| (specifications as | | | | | |
| solids content) | | | | | |
| BALANCE ® RCF | 0.1 to 10 | 0.2 to 8.0 | 0.5 to 7.0 | 0.6 to 4.0 | 0.7 to 5.0 |
| (specifications as | | | | | |
| solids content) | | | | | |
| Water | 50 to 95 | 50 to 95 | 60 to 90 | 60 to 90 | 65 to 85 |
| Misc | add 100 | add 100 | add 100 | add 100 | add 100 |

| | Formula | Formula | Formula | Formula | Formula |
|---|---|---|---|---|---|
| | 31 | 32 | 33 | 34 | 35 |
| Copolymer a) | 0.1 to 10 | 0.2 to 8.0 | 0.5 to 7.0 | 0.6 to 4.0 | 0.7 to 5.0 |
| Copolymer b) | 0.1 to 10 | 0.2 to 8.0 | 0.5 to 7.0 | 0.6 to 4.0 | 0.7 to 5.0 |
| Polyvinylpyrrolidone | 1.0 to 10 | 2.0 to 8.5 | 2.0 to 8.5 | 3.0 to 7.0 | 3.0 to 7.0 |
| Water | 50 to 95 | 50 to 95 | 60 to 90 | 60 to 90 | 65 to 85 |
| Misc | add 100 | add 100 | add 100 | add 100 | add 100 |

| | Formula | Formula | Formula | Formula | Formula |
|---|---|---|---|---|---|
| | 31a | 32a | 33a | 34a | 35a |
| Acrylates/Neodecanoate | 0.1 to 10 | 0.2 to 8.0 | 0.5 to 7.0 | 0.6 to 4.0 | 0.7 to 5.0 |
| Crosspolymer | | | | | |
| Acrylates/Ceteareth-20 | 0.1 to 10 | 0.2 to 8.0 | 0.5 to 7.0 | 0.6 to 4.0 | 0.7 to 5.0 |
| Methacrylate Crosspolymer | | | | | |
| Polyvinylpyrrolidone | 1.0 to 10 | 2.0 to 8.5 | 2.0 to 8.5 | 3.0 to 7.0 | 3.0 to 7.0 |
| Water | 50 to 95 | 50 to 95 | 60 to 90 | 60 to 90 | 65 to 85 |
| Misc | add 100 | add 100 | add 100 | add 100 | add 100 |

| | Formula | Formula | Formula | Formula | Formula |
|---|---|---|---|---|---|
| | 31b | 32b | 33b | 34b | 35b |
| Aculyn ® 38 | 0.1 to 10 | 0.2 to 8.0 | 0.5 to 7.0 | 0.6 to 4.0 | 0.7 to 5.0 |
| (specifications as | | | | | |
| solids content) | | | | | |
| BALANCE ® RCF | 0.1 to 10 | 0.2 to 8.0 | 0.5 to 7.0 | 0.6 to 4.0 | 0.7 to 5.0 |
| (specifications as | | | | | |
| solids content) | | | | | |
| Polyvinylpyrrolidone | 1.0 to 10 | 2.0 to 8.5 | 2.0 to 8.5 | 3.0 to 7.0 | 3.0 to 7.0 |
| Water | 50 to 95 | 50 to 95 | 60 to 90 | 60 to 90 | 65 to 85 |
| Misc | add 100 | add 100 | add 100 | add 100 | add 100 |

| | Formula | Formula | Formula | Formula | Formula |
|---|---|---|---|---|---|
| | 36 | 37 | 38 | 39 | 40 |
| Copolymer a) | 0.1 to 10 | 0.2 to 8.0 | 0.5 to 7.0 | 0.6 to 4.0 | 0.7 to 5.0 |
| Copolymer b) | 0.1 to 10 | 0.2 to 8.0 | 0.5 to 7.0 | 0.6 to 4.0 | 0.7 to 5.0 |
| Vinylpyrrolidone/ | 1.0 to 10 | 2.0 to 8.5 | 2.0 to 8.5 | 3.0 to 7.0 | 3.0 to 7.0 |
| Vinylacetate-Copolymer | | | | | |
| Water | 50 to 95 | 50 to 95 | 60 to 90 | 60 to 90 | 65 to 85 |
| Misc | add 100 | add 100 | add 100 | add 100 | add 100 |

| | Formula | Formula | Formula | Formula | Formula |
|---|---|---|---|---|---|
| | 36a | 37a | 38a | 39a | 40a |
| Acrylates/Neodecanoate | 0.1 to 10 | 0.2 to 8.0 | 0.5 to 7.0 | 0.6 to 4.0 | 0.7 to 5.0 |
| Crosspolymer | | | | | |
| Acrylates/Ceteareth-20 | 0.1 to 10 | 0.2 to 8.0 | 0.5 to 7.0 | 0.6 to 4.0 | 0.7 to 5.0 |
| Methacrylate Crosspolymer | | | | | |
| Vinylpyrrolidone/ | 1.0 to 10 | 2.0 to 8.5 | 2.0 to 8.5 | 3.0 to 7.0 | 3.0 to 7.0 |
| Vinylacetate-Copolymer | | | | | |
| Water | 50 to 95 | 50 to 95 | 60 to 90 | 60 to 90 | 65 to 85 |
| Misc | add 100 | add 100 | add 100 | add 100 | add 100 |

| | Formula | Formula | Formula | Formula | Formula |
|---|---|---|---|---|---|
| | 36b | 37b | 38b | 39b | 40b |
| Aculyn ® 38 | 0.1 to 10 | 0.2 to 8.0 | 0.5 to 7.0 | 0.6 to 4.0 | 0.7 to 5.0 |
| (specifications | | | | | |
| as solids content) | | | | | |
| BALANCE ® RCF | 0.1 to 10 | 0.2 to 8.0 | 0.5 to 7.0 | 0.6 to 4.0 | 0.7 to 5.0 |
| (specifications as | | | | | |
| solids content) | | | | | |
| Vinylpyrrolidone/ | 1.0 to 10 | 2.0 to 8.5 | 2.0 to 8.5 | 3.0 to 7.0 | 3.0 to 7.0 |
| Vinylacetate-Copolymer | | | | | |
| Water | 50 to 95 | 50 to 95 | 60 to 90 | 60 to 90 | 65 to 85 |
| Misc | add 100 | add 100 | add 100 | add 100 | add 100 |

| | Formula | Formula | Formula | Formula | Formula |
|---|---|---|---|---|---|
| | 41 | 42 | 43 | 44 | 45 |
| Copolymer a) | 0.1 to 10 | 0.2 to 8.0 | 0.5 to 7.0 | 0.6 to 4.0 | 0.7 to 5.0 |
| Copolymer b) | 0.1 to 10 | 0.2 to 8.0 | 0.5 to 7.0 | 0.6 to 4.0 | 0.7 to 5.0 |
| Carbomer | 0.02 to 3.0 | 0.05 to 2.0 | 0.05 to 1.5 | 0.2 to 1.0 | 0.4 to 0.8 |
| Water | 50 to 95 | 50 to 95 | 60 to 90 | 60 to 90 | 65 to 85 |
| Misc | add 100 | add 100 | add 100 | add 100 | add 100 |

| | Formula | Formula | Formula | Formula | Formula |
|---|---|---|---|---|---|
| | 41a | 42a | 43a | 44a | 45a |
| Acrylates/Neodecanoate | 0.1 to 10 | 0.2 to 8.0 | 0.5 to 7.0 | 0.6 to 4.0 | 0.7 to 5.0 |
| Crosspolymer | | | | | |
| Acrylates/Ceteareth-20 | 0.1 to 10 | 0.2 to 8.0 | 0.5 to 7.0 | 0.6 to 4.0 | 0.7 to 5.0 |
| Methacrylate Crosspolymer | | | | | |
| Carbomer | 0.02 to 3.0 | 0.05 to 2.0 | 0.05 to 1.5 | 0.2 to 1.0 | 0.4 to 0.8 |
| Water | 50 to 95 | 50 to 95 | 60 to 90 | 60 to 90 | 65 to 85 |
| Misc | add 100 | add 100 | add 100 | add 100 | add 100 |

| | Formula | Formula | Formula | Formula | Formula |
|---|---|---|---|---|---|
| | 41b | 42b | 43b | 44b | 45b |
| Aculyn ® 38 | 0.1 to 10 | 0.2 to 8.0 | 0.5 to 7.0 | 0.6 to 4.0 | 0.7 to 5.0 |
| (specifications as | | | | | |
| solids content) | | | | | |
| BALANCE ® RCF | 0.1 to 10 | 0.2 to 8.0 | 0.5 to 7.0 | 0.6 to 4.0 | 0.7 to 5.0 |
| (specifications as | | | | | |
| solids content) | | | | | |
| Carbomer | 0.02 to 3.0 | 0.05 to 2.0 | 0.05 to 1.5 | 0.2 to 1.0 | 0.4 to 0.8 |
| Water | 50 to 95 | 50 to 95 | 60 to 90 | 60 to 90 | 65 to 85 |
| Misc | add 100 | add 100 | add 100 | add 100 | add 100 |

| | Formula | Formula | Formula | Formula | Formula |
|---|---|---|---|---|---|
| | 46 | 47 | 48 | 49 | 50 |
| Copolymer a) | 0.1 to 10 | 0.2 to 8.0 | 0.5 to 7.0 | 0.6 to 4.0 | 0.7 to 5.0 |
| Copolymer b) | 0.1 to 10 | 0.2 to 8.0 | 0.5 to 7.0 | 0.6 to 4.0 | 0.7 to 5.0 |
| PEG-40 Hydrogenated | 0.05 to 1.5 | 0.1 to 1.0 | 0.2 to 0.9 | 0.3 to 0.8 | 0.4 to 0.6 |
| Castor Oil | | | | | |
| Water | 50 to 95 | 50 to 95 | 60 to 90 | 60 to 90 | 65 to 85 |
| Misc | add 100 | add 100 | add 100 | add 100 | add 100 |

| | Formula | Formula | Formula | Formula | Formula |
|---|---|---|---|---|---|
| | 46a | 47a | 48a | 49a | 50a |
| Acrylates/Neodecanoate | 0.1 to 10 | 0.2 to 8.0 | 0.5 to 7.0 | 0.6 to 4.0 | 0.7 to 5.0 |
| Crosspolymer | | | | | |
| Acrylates/Ceteareth-20 | 0.1 to 10 | 0.2 to 8.0 | 0.5 to 7.0 | 0.6 to 4.0 | 0.7 to 5.0 |
| Methacrylate Crosspolymer | | | | | |
| PEG-40 Hydrogenated | 0.05 to 1.5 | 0.1 to 1.0 | 0.2 to 0.9 | 0.3 to 0.8 | 0.4 to 0.6 |
| Castor Oil | | | | | |
| Water | 50 to 95 | 50 to 95 | 60 to 90 | 60 to 90 | 65 to 85 |
| Misc | add 100 | add 100 | add 100 | add 100 | add 100 |

| | Formula | Formula | Formula | Formula | Formula |
|---|---|---|---|---|---|
| | 46b | 47b | 48b | 49b | 50b |
| Aculyn ® 38 | 0.1 to 10 | 0.2 to 8.0 | 0.5 to 7.0 | 0.6 to 4.0 | 0.7 to 5.0 |
| (specifications as | | | | | |
| solids content) | | | | | |
| BALANCE ® RCF | 0.1 to 10 | 0.2 to 8.0 | 0.5 to 7.0 | 0.6 to 4.0 | 0.7 to 5.0 |
| (specifications as | | | | | |
| solids content) | | | | | |
| PEG-40 Hydrogenated | 0.05 to 1.5 | 0.1 to 1.0 | 0.2 to 0.9 | 0.3 to 0.8 | 0.4 to 0.6 |
| Castor Oil | | | | | |
| Water | 50 to 95 | 50 to 95 | 60 to 90 | 60 to 90 | 65 to 85 |
| Misc | add 100 | add 100 | add 100 | add 100 | add 100 |

In the context of the present disclosure, “Misc” is understood to mean a cosmetic carrier, particularly water (unless indicated separately) and optionally additional components of styling products as described above.

The cosmetic preparation of the present disclosure can be packed in forms for temporary deformation of hair, such as hair gel, hairspray, mousse or hair wax. Packaging as hair gel is exemplary.

Hair mousse and hairsprays may require the presence of propellants. As contemplated herein, however, these propellants, in some embodiment, should not contain any or contain only minor quantities of hydrocarbons. Propane, propane/butane mixtures and dimethyl ether are suitable propellants as contemplated herein.

The present disclosure also relates to the use of disclosed cosmetic preparations for temporary deformation of keratinous fibers, such as of human hair, as well as a method for temporary deformation of keratinous fibers, such as human hair, wherein the disclosed cosmetic preparation is applied on keratinous fibers.

An additional subject of this disclosure is the use of the disclosed cosmetic preparation for improvement of the hold of temporarily deformed keratinous fibers.

### Illustrative Examples

The following hair gels were produced:

| Component/raw | INCI designation | | | |
|---|---|---|---|---|
| material | or chemical name | V1 | V2 | E1 |
| Aculyn ® 38 ¹ | Acrylates/Neodecanoate | 3.3 | | 1.65 |
| | Crosspolymer | | | |
| BALANCE ® | Acrylates/Ceteareth-20 | | 3.3 | 1.65 |
| RCF ² | Methacrylate Crosspolymer | | | |
| AMP-ULTRA | Aminomethyl Propanol | 0.3 | 0.3 | 0.3 |
| PC 2000 | | | | |
| Water | | 96.4 | 96.4 | 96.4 |
| Total | | 100 | 100 | 100 |

¹ 30 wt. % of active substance in water
² 30 wt. % of active substance in water

The quantity specifications in the above table are specified as wt. % of the respective raw material, relative to the total preparation. The polymer content in each of the preparations V1, V2 and E1 was 1.0 wt. %.

The maximum hold (N) was determined for the produced styling agents by employing a 3PB test (3-point bending test) on cleaned Kerling hair strands (mean value of 5 hair strands each):

| | V1 | V2 | E1 |
|---|---|---|---|
| Fmax | 2.2 | 2.4 | 3.3 |

The polymer combination E1 in accordance with the present disclosure demonstrated a clear overadditive, synergistic effect with regard to the maximum hold.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic preparation for temporary deformation of keratinous fibers, which comprises:
a) at least one crosslinked anionic copolymer (a), which
comprises at least one structural unit according to formula (a1) 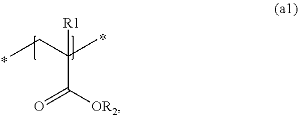
wherein R1 denotes —H or —CH₃ and R2 denotes —H or —CH₃ or —CH₂CH₃ or —CH₂CH₂CH₃ or —CH(CH₃)₂,
and comprises at least one additional structural unit according to the formula (a2) differing from the structural unit (a1) 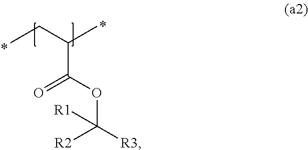
wherein R1 and R2, independently from each other, denote —H or —CH₃ or —CH₂CH₃ or —CH₂CH₂CH₃ or —CH(CH₃)₂ and R3 denotes a saturated or unsaturated, straight-chain or branched C₆₋₂₂ hydrocarbon radical
and
b) at least one crosslinked acrylate copolymer (b) differing from the copolymer (a), which comprises at least one of the following structural units (b1) and (b2): 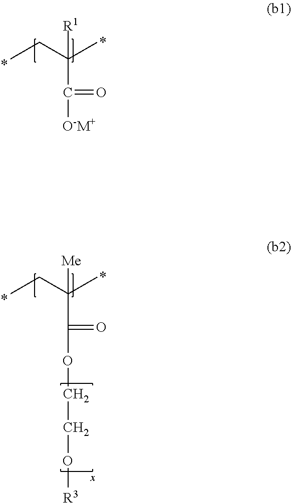
where
R¹ denotes a hydrogen atom or a methyl group,
R³ denotes a (C₈-C₃₀)-alkyl group,
M⁺ denotes a physiologically tolerated cation and
x denotes an integer from 5 to about 35.

2. The cosmetic preparation according to claim 1, wherein the copolymer (a) comprises at least one of the following monomer units:
at least one (meth)acrylic acid unit,
at least one vinyl neodecanoate unit

3. The cosmetic preparation according to claim 1, wherein the preparation comprises the copolymer (a) in an amount of from about 0.1 to about 10 wt. %.

4. The cosmetic preparation according to claim 1, wherein the radical R¹ in the structural unit (b1) denotes a methyl group.

5. The cosmetic preparation according to claim 1, wherein the x in the structural unit (b2) of the crosslinked acrylate copolymer (b) denotes an integer from about 10 to about 24.

6. The cosmetic preparation according to claim 1, wherein R³ in the structural unit (b2) of the crosslinked acrylate copolymer (b) denotes a (C₁₂-C₂₀)-alkyl group.

7. The cosmetic preparation according to claim 1, wherein the preparation comprises the anionic copolymer (b) in an amount of from about 0.1 to about 10 wt. %.

8. The cosmetic preparation according to claim 1, wherein the preparation comprises water in an amount of from about 50 to about 95 wt. %.

9. A use of the cosmetic preparation according to claim 1 for temporary deformation of keratinous fibers.

10. A method for temporary deformation of keratinous fibers wherein the cosmetic preparation according to claim 1 is applied on keratinous fibers.

11. The cosmetic preparation according to claim 1, wherein the preparation comprises the copolymer (a) in an amount of from about 0.5 to about 7.0 wt. %.

12. The cosmetic preparation according to claim 1, wherein the preparation comprises the copolymer (a) in an amount of from about 0.7 to about 5.0 wt. %.

13. The cosmetic preparation according to claim 1, wherein the x in the structural unit (b2) of the crosslinked acrylate copolymer (b) denotes an integer from about 16 to about 22.

14. The cosmetic preparation according to claim 1, wherein the x in the structural unit (b2) of the crosslinked acrylate copolymer (b) denotes an integer of about 20.

15. The cosmetic preparation according to claim 1, wherein R³ in the structural unit (b2) of the crosslinked acrylate copolymer (b) denotes a (C₁₄-C₂₀)-alkyl group.

16. The cosmetic preparation according to claim 1, wherein R³ in the structural unit (b2) of the crosslinked acrylate copolymer (b) denotes a (C₁₆-C₁₈)-alkyl group.

17. The cosmetic preparation according to claim 1, wherein the preparation comprises the anionic copolymer (b) in an amount of from about 0.5 to about 7.0 wt. %.

18. The cosmetic preparation according to claim 1, wherein the preparation comprises the anionic copolymer (b) in an amount of from about 0.7 to about 5.0 wt. %.

19. The cosmetic preparation according to claim 1, wherein the preparation comprises water in an amount of from about 60 to about 90 wt. %.

20. The cosmetic preparation according to claim 1, wherein the preparation comprises water in an amount of from about 65 to about 85 wt. %.
